(19) United States
(12) Patent Application Publication
(10) Pub. No.: US 2017/0265142 A1
(43) Pub. Date: Sep. 14, 2017

(54) SENSOR DATA EXTRACTION SYSTEM, SENSOR DATA EXTRACTION METHOD, AND COMPUTER-READABLE STORAGE MEDIUM HAVING SENSOR DATA EXTRACTION PROGRAM STORED THEREON

(71) Applicant: CASIO COMPUTER CO., LTD., Tokyo (JP)

(72) Inventor: Kazuo URA, Tokyo (JP)

(73) Assignee: CASIO COMPUTER CO., LTD., Tokyo (JP)

(21) Appl. No.: 15/608,295

(22) Filed: May 30, 2017

Related U.S. Application Data
(62) Division of application No. 14/094,368, filed on Dec. 2, 2013.

(30) Foreign Application Priority Data
Dec. 19, 2012 (JP) 2012-276641

(51) Int. Cl. H04W 52/02; G06F 19/00; A61B 5/00; G06F 17/30

(52) U.S. Cl. CPC H04W 52/0254; G06F 17/30516; G06F 19/3481; A61B 5/6813

(57) ABSTRACT

A sensor data extraction system including at least one computation circuit; and at least one memory storing instructions. When executed by the at least one computation circuit, the at least one memory storing circuit cause the at least one computation circuit to perform operations including obtaining exercise data including position data of a human body during exercise; specifying an extraction condition for acquiring exercise data for analysis from the obtained exercise data, the extraction condition relating to a meteorological change; obtaining environmental information including meteorological information from environmental information stored in a storage, the environmental information being associated with time data; extracting environmental information from the obtained environmental information, based on the specified extraction condition relating to the meteorological change; and acquiring the exercise data for analysis based on the associated time data of the extracted environmental information.

### CROSS-REFERENCE TO RELATED APPLICATION

This application is a Divisional application of U.S. Ser. No. 14/094,368, filed on Dec. 2, 2013, which is based upon and claims the benefit of priority from prior Japanese Patent Application No. 2012-276641, filed Dec. 19, 2012, the entire contents of both of which are incorporated herein by reference.

### BACKGROUND OF THE INVENTION

1. Field of the Invention

The present invention relates to a sensor data extraction system, a sensor data extraction method, and a computer-readable storage medium having a sensor data extraction program stored thereon. Specifically, the present invention relates to a sensor data extraction system, a sensor data extraction method, and a computer-readable storage medium having a sensor data extraction program stored thereon which are effectively applicable in the determination of the status of an exercise such as running and walking.

2. Description of the Related Art

In recent years, because of rising health consciousness, more and more people are performing daily exercises, such as running, walking and cycling, to maintain their wellness or improve their health condition. These people are highly conscious of and interested in measuring and recording their own health conditions and exercise status by using numerical values or data. Currently, various products and technologies responding to this demand have been developed, by which users can grasp their own health conditions and exercise status by measuring and recording their footstep counts, movement distances, pulsations (heart rates), calorie consumption amounts, etc.

For example, a technology is known in which data detected by an exercise status sensing device including an acceleration sensor is transferred to an analysis/display device to analyze an exercise status and report the result to the user, as described in Japanese Patent Application Laid-Open (Kokai) Publication No. 2005-160726.

In the technology described above, a method has been adopted in which data detected by a sensor included in an exercise status sensing device is constantly stored in a memory and all the stored data is transferred to an analysis/display device to analyze an exercise status. Here, in recent years, with the development of sensor technology, the detection capability of a sensor for detecting an exercise status has been significantly improved, and accordingly the amount of data to be detected by the sensor has increased. For example, a sensor device has been known as the above-described acceleration sensor which detects acceleration components in three axis directions orthogonal to each other at a cycle of several tens to several hundreds of Hz (several tens to several hundreds of times per second). Also, when the number of factors and types of human-body exercise information to be detected by the exercise status sensing device is large or the detection time is long, the amount of data to be detected by the sensor further increases.

Accordingly, there is a problem in the above-described method in that the amount of data transfers from the data exercise status sensing device to the analysis/display device increases and whereby the transfer time is increased. In this case, the power consumption also disadvantageously increases according to the transfer time. Moreover, when the amount of data to be transferred from the data exercise status sensing device to the analysis/display device is large, a memory having a large storage capacity is required to be included in the analysis/display device as the data transfer destination, which poses a problem in that the product cost increases.

### SUMMARY OF THE INVENTION

In accordance with one aspect of the present invention, there is provided a sensor data extraction system comprising: an exercise data obtaining section which obtains exercise data related to an exercise status of a human body; an extraction condition specifying section which specifies an extraction condition for extracting a portion required for analysis processing using the exercise data, from among the exercise data obtained by the exercise data obtaining section; a data extracting section which extracts exercise data matching the extraction condition from among the exercise data obtained by the exercise data obtaining section; a data transfer section which transfers the extracted exercise data from the exercise data obtaining section; and a data analyzing section which performs the analysis processing by using the exercise data transferred by the data transfer section.

In accordance with another aspect of the present invention, there is provided a sensor data extraction method comprising: a step of obtaining exercise data related to an exercise status of a human body; a step of specifying an extraction condition for extracting a portion required for analysis processing using the exercise data, from among the exercise data; a step of extracting exercise data matching the extraction condition from among the obtained exercise data; a step of transferring the extracted exercise data; and a step of performing the analysis processing by using the transferred exercise data. [0009—Claim 16] In accordance with another aspect of the present invention, there is provided a non-transitory computer-readable storage medium having stored thereon a sensor data extraction program that is executable by a computer, the program being executable by the computer to perform functions comprising: processing for obtaining exercise data related to an exercise status of a human body; processing for specifying an extraction condition for extracting a portion required for analysis processing using the exercise data, from among the exercise data; processing for extracting exercise data matching the extraction condition from among the obtained exercise data; processing for transferring the extracted exercise data; and processing for performing the analysis processing by using the transferred exercise data.

The above and further objects and novel features of the present invention will more fully appear from the following detailed description when the same is read in conjunction with the accompanying drawings. It is to be expressly understood, however, that the drawings are for the purpose of illustration only and are not intended as a definition of the limits of the invention.

### BRIEF DESCRIPTION OF THE DRAWINGS

FIG. 1 is a schematic structural view depicting an embodiment of an exercise status determination apparatus in which a sensor data extraction system according to the present invention has been applied;

FIG. 2A to FIG. 2C are schematic structural views each depicting an example of a sensor device applied in the exercise status determination apparatus according to the embodiment;

FIG. 3 is a block diagram depicting an example of the structure of a wrist-mount-type sensor device applied in the exercise status determination apparatus according to the embodiment;

FIG. 4 is a block diagram depicting an example of the structure of a chest-mount-type sensor device applied in the exercise status determination apparatus according to the embodiment;

FIG. 5 is a block diagram depicting an example of the structure of an information communication terminal applied in the exercise status determination apparatus according to the embodiment;

FIG. 6 is a block diagram depicting an example of the structure of a network server applied in the exercise status determination apparatus according to the embodiment;

FIG. 7 is a diagram depicting an example of sensor data extraction conditions applied in an exercise status determination method in the exercise status determination apparatus according to the embodiment;

FIG. 8 is a schematic view of a first flowchart depicting a first example of the exercise status determination method in the exercise status determination apparatus according to the embodiment;

FIG. 9 is a schematic view of a second flowchart depicting the first example of the exercise status determination method in the exercise status determination apparatus according to the embodiment;

FIG. 10 is a schematic view of a flowchart depicting a second example of the exercise status determination method in the exercise status determination apparatus according to the embodiment;

FIG. 11 is a schematic view of a flowchart depicting a third example of the exercise status determination method in the exercise status determination apparatus according to the embodiment;

FIG. 12 is a schematic view depicting an example of a movement route of a user that serves as a target for sensor data extraction processing applied in the exercise status determination method according to the embodiment;

FIG. 13A to FIG. 13D are schematic views each depicting sensor data and the like obtained in the movement route serving as a target for the sensor data extraction processing according to the embodiment and the extraction points of the data;

FIG. 14 is a schematic view depicting a display example of analysis data and the like displayed on a user terminal or the like applied in the exercise status determination apparatus according to the embodiment; and

FIG. 15 is a schematic structural view depicting a modification example of the exercise status determination apparatus according to the embodiment.

### DETAILED DESCRIPTION OF THE PREFERRED EMBODIMENTS

Hereafter, embodiments of a sensor data extraction system, a sensor data extraction method, and a sensor data extraction program according to the present invention are described in detail. In the following description, a case is described in which the present invention is applied to an exercise status determination apparatus that determines an exercise status when a user performs an exercise such as running or walking.

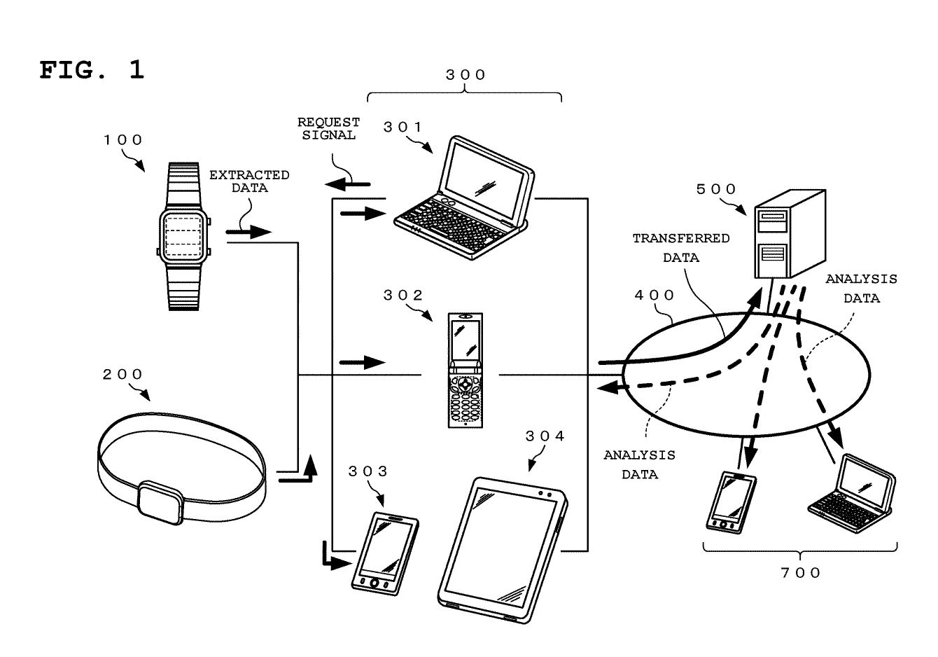

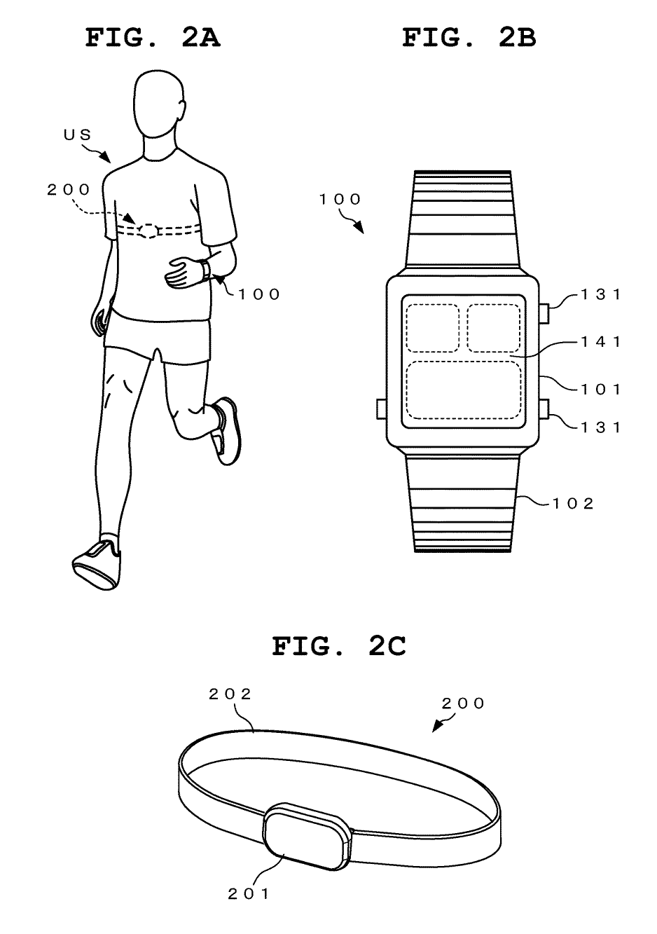

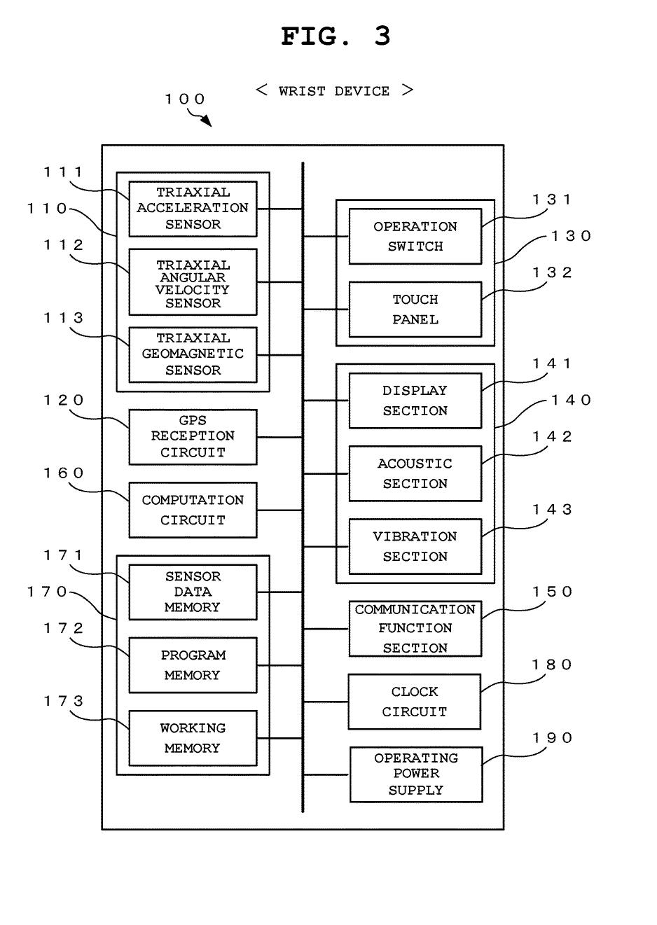

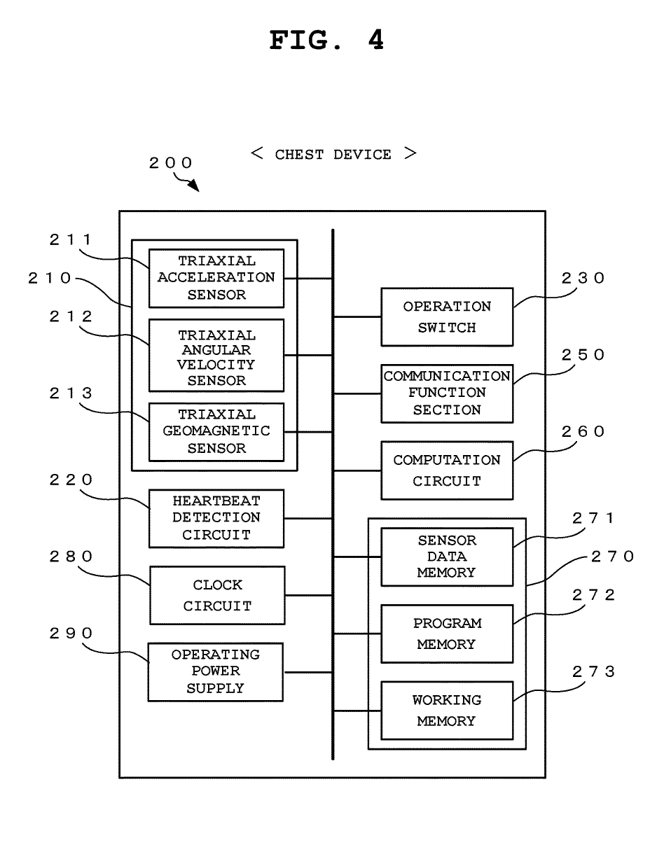

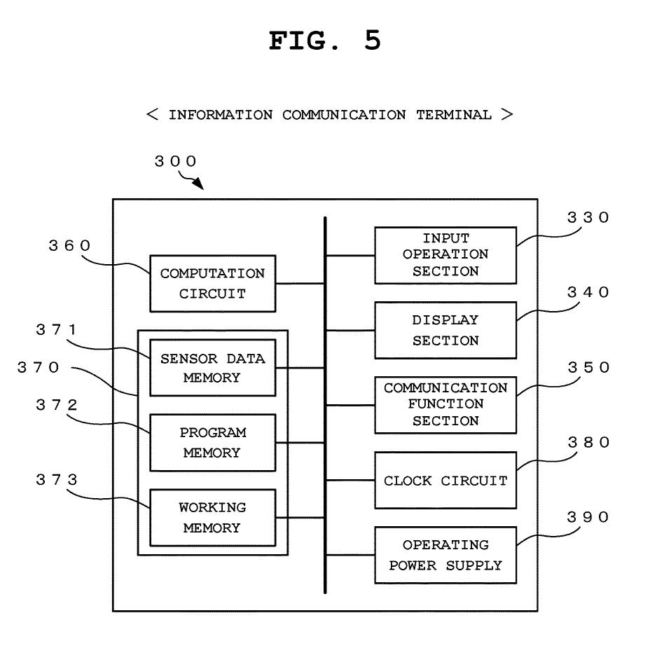

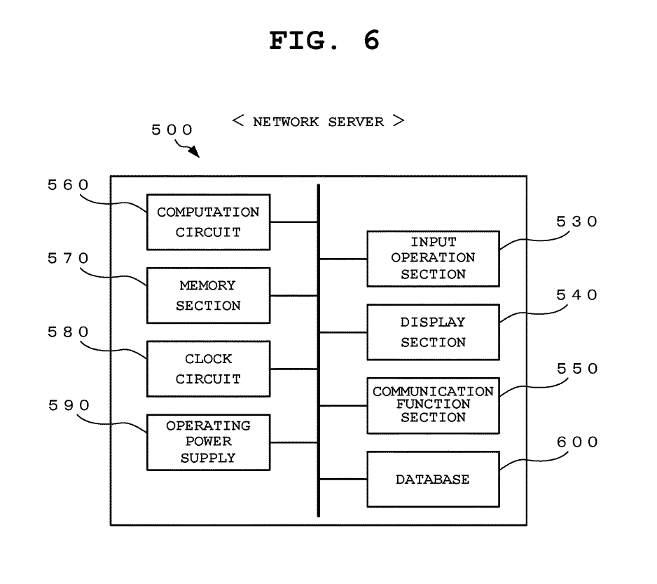

(Exercise Status Determination Apparatus) FIG. 1 is a schematic structural view depicting an embodiment of an exercise status determination apparatus in which a sensor data extraction system according to the present invention has been applied, and FIG. 2A to FIG. 2C are schematic structural views each depicting an example of a sensor device applied in the exercise status determination apparatus according to the present embodiment. FIG. 3 is a block diagram depicting an example of the structure of a wrist-mount-type sensor device applied in the exercise status determination apparatus according to the present embodiment, and FIG. 4 is a block diagram depicting an example of the structure of a chest-mount-type sensor device applied in the exercise status determination apparatus according to the present embodiment. FIG. 5 is a block diagram depicting an example of the structure of an information communication terminal applied in the exercise status determination apparatus according to the present embodiment, and FIG. 6 is a block diagram depicting an example of the structure of a network server applied in the exercise status determination apparatus according to the present embodiment.

The exercise status determination apparatus according to the present embodiment mainly includes a wrist-mount-type sensor device (hereinafter referred to as a “wrist device” for convenience of explanation) 100 or a chest-mount-type sensor device (hereinafter referred to as a “chest device” for convenience of explanation) 200 which are worn on the body of a user US who is a measurement subject, an information communication terminal 300, a network 400, a data processing device such as a network server 500, and a user terminal 700, as depicted in FIG. 1 and FIG. 2A to FIG. 2C.

(Wrist Device 100)

The wrist device 100 is a wristwatch-type or a wristband-type sensor device that is worn on a wrist of the user US, as depicted in FIGS. 2A and 2B. The wrist device 100 has an outer appearance structure mainly including a device body 101 which detects the exercise status and the position of the user US and provides predetermined information to the user US, and a band section 102 that is wound around a wrist of the user US so as to mount the device body 101 on the wrist.

Specifically, the wrist device 100 mainly includes, for example, a sensor section 110, a GPS reception circuit 120, an input interface section 130, an output interface section 140, a communication function section 150, a computation circuit 160, a memory section 170, a clock circuit 180, and an operating power supply 190, as depicted in FIG. 3.

The sensor section 110 is a motion sensor for detecting a motion of a human body (in particular, the swing of arms, the tilting status of the wrist device 100, etc.). This sensor section 110 has, for example, a triaxial acceleration sensor 111, a triaxial angular velocity sensor (a gyro sensor) 112, and a triaxial geomagnetic sensor (an electronic compass) 113, as depicted in FIG. 3. The triaxial acceleration sensor 111 detects a ratio of change in operation speed (acceleration) during the exercise of the user US and outputs acceleration data thereof. Here, acceleration data in three axis directions orthogonal to each other is outputted. The triaxial angular velocity sensor 112 detects a change in a motion direction (angular velocity) during the exercise of the user and outputs angular velocity data thereof. Here, angular velocity data in three axis directions orthogonal to each other is outputted. The triaxial geomagnetic sensor 113 detects the magnetic field of earth and outputs geomagnetic data thereof or directional data indicating the horizontal and vertical directions of the wrist device 100. Here, geomagnetic data in three axis directions orthogonal to each other is outputted. Sensor data obtained by detection by these various sensors 111 to 113 (acceleration data, angular velocity data, and geomagnetic data; exercise data) is associated with time data defined by the clock circuit 180 described below, and stored in a predetermined storage area of a sensor data storage memory 171 of the memory section 170 described below.

The GPS reception circuit 120 receives electric waves from a plurality of GPS (Global Positioning System) satellites via a GPS antenna (omitted in the drawing) so as to detect a geographic position based on latitude and longitude information and output position data thereof. This GPS reception circuit 120 uses a Doppler-shift effect of electric waves from the GPS satellites to detect the movement speed of the user US and output movement speed data thereof. As with the sensor data described above, GPS data (exercise data) including these position data and movement speed data is associated with time data defined by the clock circuit 180, and stored in a predetermined storage area of the sensor data storage memory 171 of the memory section 170. In the GPS reception circuit 120, in addition to the position data based on the above latitude and longitude information, altitude data regarding that position can be obtained. However, with the accuracy and technological specifications of present GPS reception signals, altitude data has a large error, and sufficiently practical altitude information cannot be obtained. Therefore, altitude data is not obtained in the present embodiment, or not used for subsequent analysis processing even if obtained.

The input interface section 130 has, for example, an operation switch 131 and a touch panel 132, as depicted in FIG. 3. The operation switch 131 is, for example, a press-button-type switch provided projecting to a side surface of the device body 101 as depicted in FIG. 2B, which is used for various input operations such as an operation for controlling a sensing action in various sensors provided on the above-described sensor section 110 and an operation for setting an item to be displayed on a display section 141.

The touch panel 132 is arranged on the front surface side (view field side) of the display section 141 of the output interface section 140 described below, or is integrally formed on the front surface side of the display section 141. With a touch operation on an area corresponding to information displayed on the display section 141, a function corresponding to the information is selectively performed. Note that functions to be achieved by the touch panel 132 may be equivalent to functions that are achieved by the operation switch 131 described above, or may be functions unique to input operations by the touch panel 132.

As will be described further below, in addition to the above-described operation for the functions, the input interface section 130 may be used for an operation for inputting and setting an extraction condition in data extraction processing that is performed when sensor data and GPS data are transferred from the wrist device 100 to the information communication terminal 300. Also, the input interface section 130 may be structured to include only one of the operation switch 131 and the touch panel 132.

The output interface section 140 has, for example, the display section 141, an acoustic section 142, and a vibration section 143, as depicted in FIG. 3. The display section 141 has a display panel of, for example, a liquid-crystal type capable of color or monochrome display or a light-emitting-element-type such as an organic EL (Electro Luminescence) element, and displays at least sensor data detected by the sensor section 110 described above, GPS data detected by the GPS reception circuit 120, various exercise information generated based on these sensor data and GPS data, time information such as a current time, or the like. The output interface section 140 may display sensor data and heartbeat data transmitted from the chest device 200 described below, various exercise information generated based on these sensor data and heartbeat data, and the like. Note that modes for displaying various types of information by the display section 141 are arbitrarily set by operating the operation switch 131 or the touch panel 132 described above.

The acoustic section 142 has an acoustic device such as a buzzer or a loudspeaker. By generating sound information such as a predetermined timbre, sound pattern, and voice message, the acoustic section 142 aurally provides or reports various information to the user US. The vibration section 143 has a vibration device (vibrator) such as a vibration motor or a vibrator. By generating vibration information such as a predetermined vibration pattern and its intensity, the vibration section 143 tactually provides or reports various information to the user US. The output interface section 140 may have a structure including, for example, at least one of the display section 141, the acoustic section 142, and the vibration section 143 described above. Note that the output interface section 140 preferably has a structure including at least one of the display section 141 and the acoustic section 142 for the case where specific information such as numerical value information is provided to the user US.

The communication function section 150 functions as an interface when sensor data obtained by the sensor section 110 and GPS data obtained by the GPS reception circuit 120 (hereinafter collectively referred to as “sensor data and the like”) are transmitted to the information communication terminal 300 described below. The communication function section 150 also functions as an interface with the chest device 200, which will be described further below, when a synchronizing signal for the synchronization of time data associated with sensor data, heartbeat data, and the like obtained by the chest device 200 is transmitted. Moreover, the communication function section 150 may function as an interface when sensor data, heartbeat data, and the like obtained by the chest device 200 described below is received. Note that, as a method for transferring or transmitting and receiving sensor data, a synchronizing signal, and the like between the wrist device 100 and the information communication terminal 300 or the chest device 200 via the communication function section 150, various wireless communication methods and wired communication methods via a communication cable can be adopted.

In a case where the above-described sensor data and the like are transferred via a wireless communication method, for example, Bluetooth (registered trademark), which is short-range wireless communication standards for digital devices, or Bluetooth (registered trademark) low energy (LE) developed as a low-power-consumption-type of the above-described communication standards can be favorably adopted. By this wireless communication method being adopted, data transmission can be favorably performed even with small electric power generated by using an energy harvesting technology or the like as the operating power supply 190 described below.

The memory section 170 mainly has, for example, a sensor data storage memory (hereinafter referred to as a “sensor data memory”) 171, a program storage memory (hereinafter referred to as a “program memory”) 172, and a work data storage memory (hereinafter referred to as a “working memory”) 173, as depicted in FIG. 3.

The sensor data memory 171 has a non-volatile memory for storing sensor data and the like obtained by the sensor section 110 and the GPS reception circuit 120 described above in association with each other in a predetermined storage area. The program memory 172 has stored therein a control program for performing a predetermined operation in each section, such as a sensing operation in the sensor section 110 and the GPS reception circuit 120 and a data transmitting operation in the communication function section 150, and an algorithm program for extracting sensor data and the like satisfying a predetermined extraction condition from the above-described sensor data and the like. The working memory 173 temporarily stores various data for use in executing the control program and the algorithm program and various data generated thereby. The sensor data memory 171 may be partially or entirely a removable storage medium such as a memory card, and may be structured to be attachable to and removable from the wrist device 100.

The computation circuit 160, which is a computation device such as a CPU (Central Processing Unit) or a MPU (microprocessor), executes a predetermined control program stored in the program memory 172 based on an operation clock generated by the clock circuit 180 described below, and thereby controls various operations such as a sensing operation by the various sensors 111 to 113 of the sensor section 110 and the GPS reception circuit 120, an information providing operation by the output interface section 140, and a data transmission operation by the communication function section 150. The computation circuit 160 also executes a predetermined algorithm program stored in the program memory 172. As a result, in the computation circuit 160, an operation of extracting sensor data and the like matching a desired extraction condition from sensor data and the like obtained by the sensor section 110 and the GPS reception circuit 120 is executed. The control program and the algorithm program to be executed in the computation circuit 160 may be incorporated in advance in the computation circuit 160.

The clock circuit 180 has an oscillator that generates a base clock and, based on the base clock, generates an operation clock that defines the operation timing of each component of the wrist device 100, a synchronizing signal for synchronizing time data with the chest device 200 and the information communication terminal 300, time data that indicates a current time, and the like. Also, the clock circuit 180 clocks the timing of obtaining sensor data and the like in the sensor section 110 and the GPS reception circuit 120 and outputs time data thereof. The time data is associated with the obtained sensor data and the like and stored in the sensor data memory 171. Also, the time data is displayed on the display section 141 of the output interface section 140, whereby the current time and the like are provide to the user US.

The operating power supply 190 supplies driving electric power to each component inside the device body 101 of the wrist device 100. As the operating power supply 190, for example, a primary battery such as a commercially-available coin-shaped battery or button-shaped battery or a secondary battery such as a lithium-ion battery or a nickel-metal-hydride battery can be applied. In addition, it is possible to apply a power supply by an energy harvest technology for generating electricity by energy such as vibrations, light, heat or electro-magnetic waves. In a case where the wrist device 100 has a structure where sensor data and the like are transferred by a wired communication method to the information communication terminal 300, a configuration may be adopted in which, by the wrist device 100 being connected to the information communication terminal 300 via a communication cable, driving electric power is supplied from the information communication terminal 300 to charge the secondary battery of the operating power supply 190.

(Chest Device 200)

The chest device 200 is a chest-mount-type sensor device that is worn on the chest of the user US, as depicted in FIG. 2A and FIG. 2C. This chest device 200 mainly includes a device body 201 which detects the exercise status and the biological information of the user US and a band section 202 that is wound around the chest of the user US to mount the device body 201 on the chest.

Specifically, the chest device 200 mainly includes, for example, a sensor section 210, a heartbeat detection circuit 220, an operation switch 230, a communication function section 250, an computation circuit 260, a memory section 270, a clock circuit 280, and an operating power supply 290, as depicted in FIG. 4. Here, descriptions of components similar to those of the wrist device 100 are simplified.

As with the wrist device 100, the sensor section 210 is a motion sensor for detecting a motion of a human body (in particular, an exercise form, a travelling direction, a stride, etc.) This sensor section 210 has, for example, a triaxial acceleration sensor 211, a triaxial angular velocity sensor 212, and a triaxial geomagnetic sensor 213, as depicted in FIG. 4. Sensor data obtained by detection by these various sensors 211 to 213 (acceleration data, angular velocity data, and geomagnetic data; exercise data) is associated with time data defined by the clock circuit 280 described below, and stored in a predetermined storage area of a sensor data storage memory 271 of the memory section 270 described below.

The heartbeat detection circuit 220, which is provided on the inner surface side (human body side) of the band section 202 of the chest device 200, is connected to an electrode (omitted in the drawings) placed in close direct contact with the chest of the user US, and detects a heartbeat from a change in electrocardiographic signals outputted from the electrode. As with the sensor data described above, the detected heartbeat data (exercise data and biological information) is associated with time data defined by the clock circuit 280 and stored in a predetermined storage area of the sensor data memory 271 of the memory section 270.

The operation switch 230 is an input interface having at least a power supply switch. By the operation switch 230 being operated by the user US, the status of supply (supply or interrupt) of driving electric power from the operating power supply 290 to each component is controlled so as to control ON/OFF of the power supply of the chest device 200. This operation switch 230 also has a sensor control key switch. By the operation switch 230 being operated by the user US, the start and stop of a sensing operation by the sensor section 210 and the heartbeat detection circuit 220 is controlled.

As with the wrist device 100, the communication function section 250 functions as an interface when sensor data obtained by the sensor section 210 and heartbeat data obtained by the heartbeat detection circuit 220 (sensor data and the like) are transmitted to the information communication terminal 300 and the wrist device 100 and when synchronization with the wrist device 100 is performed. Note that, as a method for transferring or transmitting and receiving sensor data, a synchronizing signal, and the like between the chest device 200 and the information communication terminal 300 or the wrist device 100 via the communication function section 250, various wireless communication methods and wired communication methods via a communication cable can be adopted, as in the case of the wrist device.

As with the wrist device 100, the memory section 270 mainly has a sensor data memory 271, a program memory 272, and a working memory 273. The sensor data memory 271 stores, in a predetermined storage area, sensor data and the like obtained by the sensor section 210 and the heartbeat detection circuit 220 in association with each other. The program memory 272 has stored therein a control program for performing a predetermined operation in each section, such as a sensing operation in the sensor section 210 and the heartbeat detection circuit 220 and a data transmitting operation in the communication function section 250, and an algorithm program for extracting sensor data and the like matching a predetermined extraction condition from the above-described sensor data and the like. The working memory 273 temporarily stores various data for use in executing the control program and the algorithm program, and various data generated thereby. As with the wrist device 100, the sensor data memory 271 may be partially or entirely a removable storage medium, and may be structured to be attachable to and removable from the chest device 200.

As with the wrist device 100, the computation circuit 260 executes a predetermined control program stored in the program memory 272 based on an operation clock generated by the clock circuit 280 described below, and thereby controls various operations such as a sensing operation by the various sensors 211 to 213 of the sensor section 210 and the heartbeat detection circuit 220 and a data transmission operation by the communication function section 250. The computation circuit 260 also executes a predetermined algorithm program stored in the program memory 272. As a result, in the computation circuit 260, an operation of extracting sensor data and the like matching a desired extraction condition from sensor data and the like obtained by the sensor section 210 and the heartbeat detection circuit 220 is executed. The control program and the algorithm program to be executed in the computation circuit 260 may be incorporated in advance in the computation circuit 260.

The clock circuit 280 has an oscillator that generates a base clock and, based on the base clock, generates an operation clock that defines the operation timing of each component of the chest device 200. Also, the clock circuit 280 clocks the timing of obtaining sensor data and the like in the sensor section 210 and the heartbeat detection circuit 220 and outputs time data thereof. The time data is associated with the obtained sensor data and the like and stored in the sensor data memory 271. Then, based on a synchronizing signal transmitted from the above-described wrist device 100, the synchronization of time data is performed between the chest device 200 and the wrist device 100. This synchronizing operation between the wrist device 100 and the chest device 200 may be performed in the wrist device 100 and the chest device 200 at, for example, activation timing at which the power supply is turned ON or at sensing operation start timing in the sensor sections 110 and 210. Also, it may be performed at predetermined time intervals, at arbitrary timing, or at all times.

The operating power supply 290 supplies driving electric power to each component inside the device body 201 of the chest device 200 by the operation switch 230 being operated. As the operating power supply 290, for example, a primary battery or a secondary battery can be applied. In addition, it is possible to apply a power supply by an energy harvest technology. In a case where the chest device 200 has a structure where sensor data and the like are transferred by a wired communication method to the information communication terminal 300, a configuration may be adopted in which, by the chest device 200 being connected to the information communication terminal 300 via a communication cable, driving electric power is supplied from the information communication terminal 300 to charge the secondary battery of the operating power supply 290.

(Information Communication Terminal 300)

The information communication terminal 300 has a function for connecting to a network 400 such as the Internet. As the information communication terminal 300, a network communication device having incorporated therein a web browser as viewing software, such as a notebook or desktop personal computer 301, a portable telephone 302, an advanced portable telephone (hereinafter referred to as a “smartphone”) 303, a tablet terminal 304, or a dedicated terminal (omitted in the drawing) can be adopted, as depicted in FIG. 1. In particular, in a network communication device such as the portable telephone 302, the smartphone 303, or the tablet terminal 304, a function for connecting to the network 400 and a web browser have already been included, and therefore connection can be easily made to the network 400 anywhere within a prescribed communicable range.

Specifically, the information communication terminal 300 mainly includes, for example, an input operating section 330, a display section 340, a communication function section 350, a computation circuit 360, a memory section 370, a clock circuit 380, and an operating power supply 390, as depicted in FIG. 5. Here, descriptions of components similar to those of the wrist device 100 and the chest device 200 are simplified.

The input operating section 330 is an input means, such as a keyboard, a mouse, a touch pad, a dial key, or a touch panel, annexed to the personal computer 301, the portable telephone 302, the smartphone 303, the tablet terminal 304, etc. By selecting an arbitrary icon or menu displayed on the display section 340 or pointing an arbitrary area on the screen display by using the input operating section 330, a function corresponding to this icon, menu, or area is performed.

The display section 340 has, for example, a monitor or a display panel of a liquid-crystal type or a light-emitting-element type, and displays a screen for setting various conditions and information for at least processing for extracting sensor data and the like which is performed in the wrist device 100 and the chest device 200. The display section 340 also displays a communication status and a transfer situation when sensor data and the like obtained in the wrist device 100 and the chest device 200 are transferred to the network server 500 via the network 400 described below. In a case where the information communication terminal 300 is adopted as the user terminal 700 for viewing, for example, analysis data obtained by analysis processing in the network server 500, sensor data and the like obtained in the wrist device 100 and the chest device 200, their analysis data, and specific information regarding the exercise status of the user US generated based on the analysis data are displayed on the display section 340 in the form of numerical values, a graph, a map, animation, etc. These sensor data, analysis data, and specific information which are displayed on the user terminal 700 will be described in detail further below.

The communication function section 350 functions as an interface when sensor data and the like obtained in the wrist device 100 and the chest device 200 are transmitted to the network server 500 via the network 400 described below and when analysis data and the like obtained by analysis in the network server 500 is received. Also, this communication function section 350 functions as an interface when a synchronizing signal transmitted from the wrist device 100 for the synchronization of time data between the wrist device 100 and the chest device 200 is received. Note that, as a method for transferring or transmitting and receiving sensor data, a synchronizing signal, and the like between the information communication terminal 300 and the wrist device 100 or the chest device 200 via the communication function section 350 as described above, various wireless communication methods and wired communication methods can be adopted. Also, as a connection method between the information communication terminal 300 and the network 400 when sensor data and the like are transferred by the communication function section 350 to the network server 500, for example, a wired connection method for connection via an optical fiber line network or an ADSL (Asymmetric Digital Subscriber Line) network or a wireless connection method for connection via a portable telephone network or a high-speed mobile communication network can be adopted.

The memory section 370 mainly includes a sensor data memory 371, a program memory 372, and a working memory 373, as in the case of the wrist device 100 and the chest device 200. The sensor data memory 371 has a non-volatile memory for storing sensor data and the like transferred from the wrist device 100 and the chest device 200 in association with each other in a predetermined storage area. The program memory 372 has stored therein a control program for performing a predetermined operation in each section, such as a display operation in the display section 340 and a data transmitting operation in the communication function section 350, and a control program for performing a condition setting operation for extracting sensor data and the like matching a desired extraction condition from sensor data and the like obtained by the wrist device 100 and the chest device 200. The working memory 373 temporarily stores various data for use in executing the control programs and various data generated thereby. In a case where the information communication terminal 300 is used as a user terminal for viewing analysis data and the like obtained by analysis in the network server 500, a structure may be adopted in which the memory section 370 has an analysis data storage memory (omitted in the drawing) for storing analysis data and the like received via the network 400. Also, the sensor data memory 371 may be partially or entirely a removable storage medium, and may be structured to be attachable to and removable from the information communication terminal 300, as in the case of the wrist device 100 and the chest device 200.

The computation circuit 360 executes a predetermined control program stored in the program memory 372 based on a operation clock generated in the clock circuit 380, and thereby controls an operation in each section, such as a display operation in the display section 340 and a data transmitting operation in the communication function section 350. The computation circuit 360 also executes a predetermined control program to perform a condition setting operation for extracting sensor data and the like. The control programs to be executed in the computation circuit 360 may be incorporated in advance in the computation circuit 360.

The clock circuit 380 generates an operation clock that defines the operation timing of each component of the information communication terminal 300. Then, based on a synchronizing signal transmitted from the wrist device 100, the synchronization of time data is performed between the information communication terminal 300 and the wrist device 100 or the chest device 200.

The operating power supply 390 supplies driving electric power to each component of the information communication terminal 300. In a portable telephone or a smartphone, a secondary battery such as a lithium-ion battery is adopted as the operating power supply 390. In a notebook personal computer or tablet terminal, a secondary battery such as a lithium-ion battery or a commercial alternating-current power supply is adopted. In a desktop personal computer, a commercial alternating-current power supply is adopted.

(Network 400)

The network 400 allows transmission and reception of sensor data, analysis data, and the like between the information communication terminal 300 and the network server 500. As will be described further below, a computer network where various information services such as geographic information and meteorological information are provided can be adopted as the network 400. Here, the network 400 may be a publicly-usable network such as the Internet or a network that is limitedly usable by a business enterprise, a university, or an organization specific to an area or the like.

(Network Server 500)

The network server 500 is an application server having at least a function for analyzing and processing data, which will be described further below. As depicted in FIG. 1, the network server 500 analyzes and processes sensor data and the like transferred from the information communication terminal 300 via the network 400 so as to generate analysis data and specific information regarding the exercise status of the user US. This network server 500 internally or externally includes a memory and a database for storing and accumulating sensor data and the like transferred from the information communication terminal 300, various data to be referred to in analysis and conversion processing, and the generated analysis data and specific information. Note that a computer network constituted by the network 400 and the network server 500 may use, for example, a commercial Internet cloud service or the like.

Specifically, the network server 500 includes, for example, an input operation section 530, a display section 540, a communication function section 550, a computation circuit 560, a memory section 570, a clock circuit 580, an operating power supply 590, and a database 600, as depicted in FIG. 6. Here, descriptions of components similar to those of the wrist device 100, the chest device 200, and the information communication terminal 300 are simplified.

The input operation section 530, which includes an input device such as a keyboard, a mouse, a touch pad, or a touch panel, is used to select an arbitrary icon or menu displayed on the display section 540 or to point an arbitrary area. The display section 540 has a monitor or a display panel, and displays information regarding various operations in the network server 500.

The communication function section 550 functions as an interface when sensor data and the like transferred from the information communication terminal 300 are received and when analysis data and the like obtained by analysis in the network server 500 are transmitted to the user terminal 700 (or the information communication terminal 300 or another network communication device).

The memory section 570 includes a transfer data memory that stores sensor data and the like transferred from the information communication terminal 300, a program memory that stores a control program for performing a predetermined operation in the display section 540 and the communication function section 550 and an algorithm program for performing predetermined analysis and conversion processing based on transferred sensor data and the like, and a working memory. The database 600 stores and accumulates analysis data generated by analyzing and processing sensor data and the like by the computation circuit 560, specific information regarding the exercise status of the user US, and various data to be referred to in the analysis and conversion processing.

The computation circuit 560 executes a predetermined algorithm program stored in the program memory based on an operation clock generated in the clock circuit 580, and thereby performs predetermined analysis and conversion processing based on sensor data and the like stored in the transfer data memory. As a result, in the computation circuit 560, analysis data based on the sensor data and the like and specific information regarding the exercise status of the user US are generated and stored in a predetermined storage area of the database 600. Also, by the user US accessing the network server 500 by using the user terminal 700, the computation circuit 560 reads out analysis data and specific information as necessary from the database 600 so as to generate web display data for displaying in a display format using numerical values, a graph, a map, animation, and the like on the user terminal 700. Note that the control program and the algorithm program to be executed in the computation circuit 560 may be incorporated in advance in the computation circuit 560. Also, as the operating power supply 590, a commercial alternating-current power supply is adopted.

(User Terminal 700)

The user terminal 700 is a network communication device having a structure similar to that of the information communication terminal 300. By the user US accessing the network server 500, the user terminal 700 receives web display data including analysis data and the like generated in the network server 500 via the network 400, and displays it by a web browser. As a result of this configuration, the user US can view analysis data based on sensor data and the like detected during an exercise such as running, form data regarding his or her exercise form, and related information such as geographic information and meteorological information during the exercise in a display format where these pieces of information are singly displayed or a display format where they are displayed by being coordinated with other, and thereby can analyze his or her own exercise status and reflect the analysis results in the improvement of an exercise method thereafter. Note that, as the user terminal 700, the information communication terminal 300 used for transferring sensor data and the like to the network server 500 may be directly applied, or a network communication device different from the information communication terminal 300 may be applied. That is, in the former structure, the same information communication terminal 300 can be used for transferring sensor data and the like and for viewing analysis data and the like, and therefore the user US is not required to own or hold a plurality of electronic devices and the exercise status determination apparatus according to the present embodiment can be achieved with a simple structure. On the other hand, in the latter structure, for example, the portable telephone 302 or the smartphone 303 can be used to transfer sensor data and the like, and analysis data and the like can be viewed by using a large screen of the personal computer 301 or the tablet terminal 304. Therefore, the user US can perform each operation by using an electronic device with higher usability.

Next, sensor data extraction conditions applied in an exercise status determination method in the exercise status determination apparatus according to the present embodiment are described.

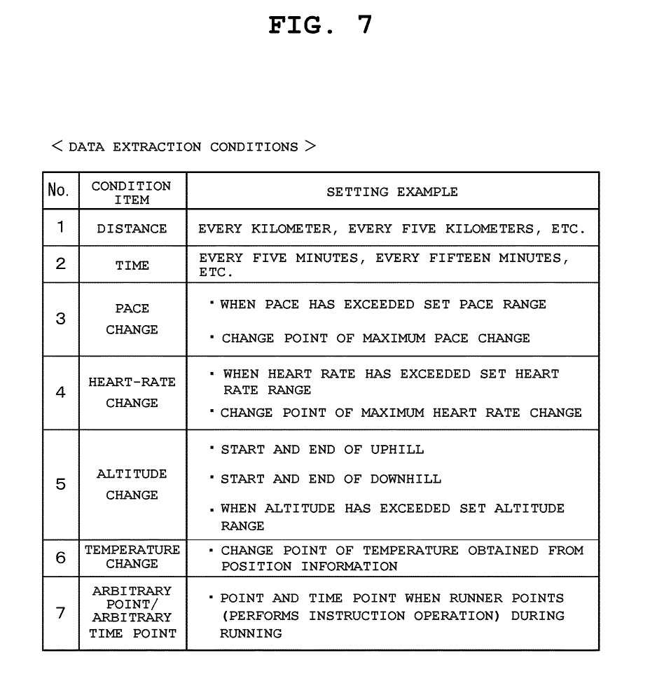

FIG. 7 is a diagram depicting an example of sensor data extraction conditions applied in the exercise status determination method in the exercise status determination apparatus according to the embodiment.

In the exercise status determination apparatus according to the present embodiment, extraction conditions such as those depicted in FIG. 7 are set. Then, from sensor data and the like obtained in the wrist device 100 and the chest device 200, sensor data and the like matching the extraction conditions are extracted and transferred to the information communication terminal 300. Specifically in the present embodiment, for example, (1) distance, (2) time, (3) pace change, (4) heart rate change, (5) altitude change, (6) temperature change, and (7) arbitrary point/arbitrary time point can be set as extraction conditions.

Specifically, (1) in the distance condition, regarding distance data calculated based on position data included in GPS data, sensor data and the like are extracted at every predetermined distance, such as every kilometer or every five kilometers. (2) In the time condition, sensor data and the like are extracted based on time data at every predetermined time intervals, such as every five minutes or every fifteen minutes.

(3) In the pace change condition, regarding movement speed data included in GPS data or speed data calculated based on position data and time data, sensor data and the like acquired at timing at which the pace has exceeded a numerical value range set in advance or the pace has extremely changed, or sensor data and the like acquired at this timing and around this timing is extracted.

(4) In the heart rate change condition, sensor data and the like acquired at timing at which heartbeat data (heart rate) has exceeded a numerical value range set in advance or the heart rate has extremely changed, or sensor data and the like acquired at this timing and around this timing is extracted.

(5) In the altitude change condition, sensor data and the like acquired at timing at which the current point has been judged to be the start (staring point) or end (ending point) of an uphill or downhill based on an altitude change or at which the altitude has exceeded a numerical value range set in advance, or sensor data and the like acquired at this timing and around this timing is extracted.

(6) In the temperature change condition, sensor data and the like acquired at timing at which a temperature change point (a simple temperature change or, for example, a change in temperature tendency such as a change from an increasing tendency to a decreasing tendency) has been observed or at which the temperature change has exceeded a numerical value range set in advance, or sensor data and the like acquired at this timing and around this timing is extracted. Here, in (5) the altitude change extraction and (6) the temperature change extraction, entire position data (latitude and longitude data) included in the GPS data is transferred from the wrist device 100 to the information communication terminal 300, and the information communication terminal 300 connects to the network 400 such as the Internet to obtain altitude information and temperature information corresponding to each piece of position data from a site or dedicated server that provides environment information such as geographic information and meteorological information, whereby altitude and temperature changing points are set. Note that the altitude information obtained from the site or the dedicated server may be corrected based on barometric pressure information in the meteorological information. Also, in a structure where the present system includes a barometric pressure sensor, the altitude information may be corrected based on sensor data of the barometric pressure sensor.

(7) In the arbitrary point/arbitrary time point extraction, by the user operating the operation switch 131 or the touch panel 132 of the wrist device 100 at an arbitrary point or time during an exercise, sensor data and the like acquired at this timing or sensor data and the like acquired at this timing and around this timing is extracted.

(Exercise Status Determination Method)

Next, the exercise status determination method in which the sensor data extracting method according to the present invention has been applied is described. Here, in the present invention, the processing procedure and processing details are varied according to an extraction condition (refer to FIG. 7) specified in the above-described processing for extracting sensor data and the like, and therefore an example is described for each type of extraction condition. Also, explanations herein are made with reference to the structure of the exercise status determination apparatus described above.

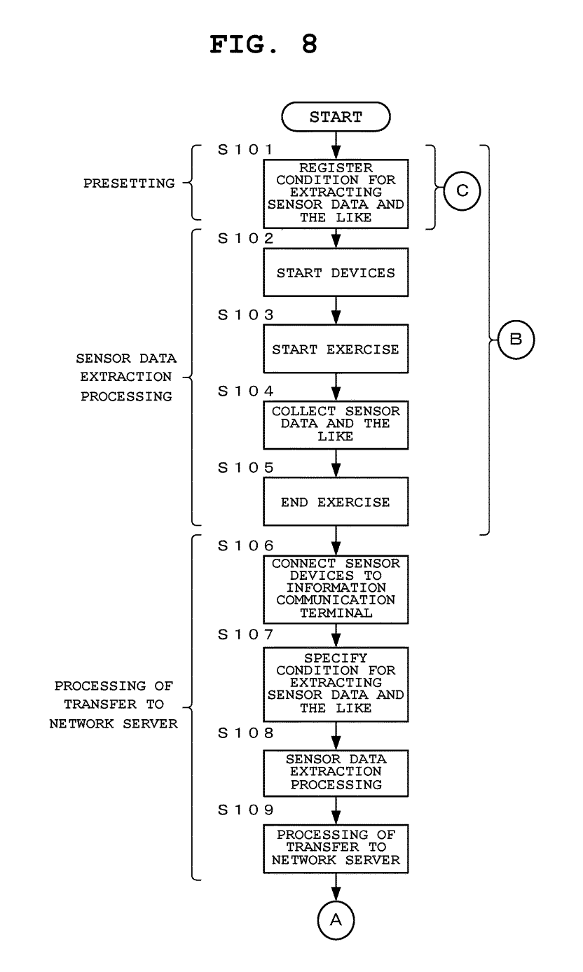

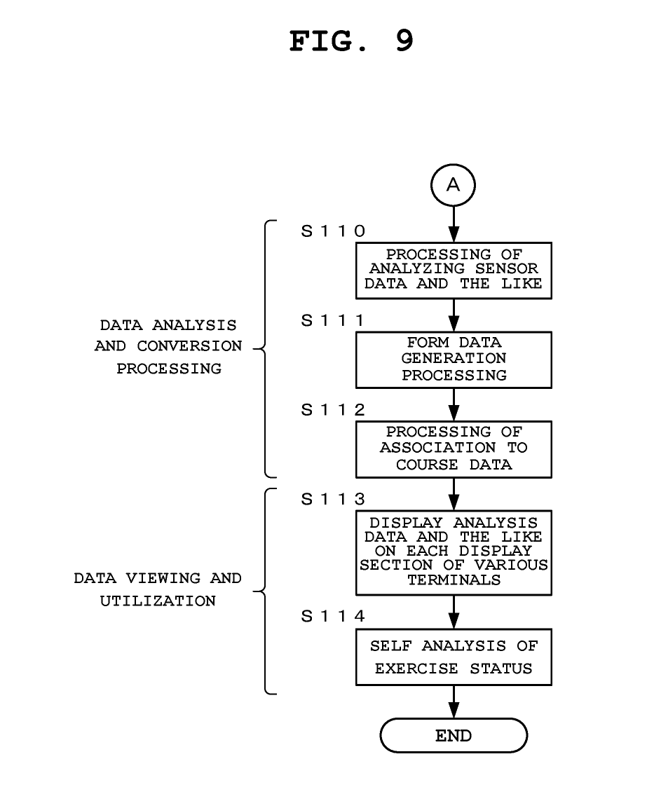

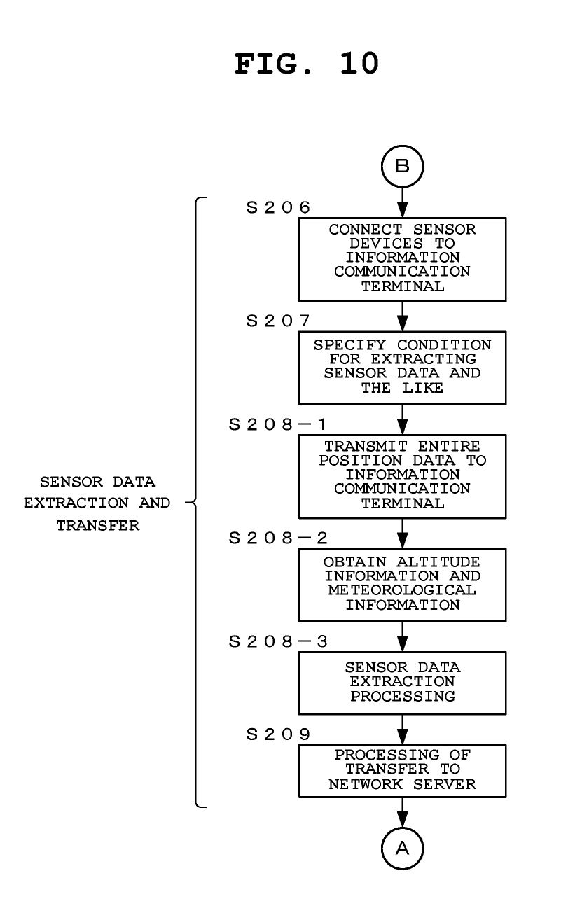

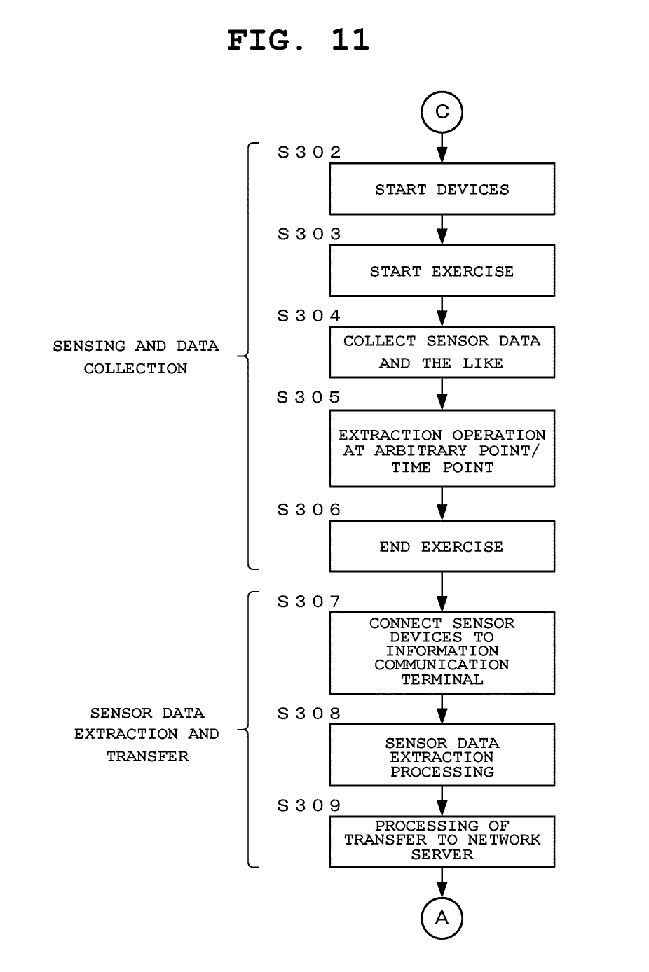

FIG. 8 and FIG. 9 are flowcharts depicting a first example of the exercise status determination method in the exercise status determination apparatus according to the present embodiment. FIG. 10 is a flowchart depicting a second example of the exercise status determination method in the exercise status determination apparatus according to the present embodiment. FIG. 11 is a flowchart depicting a third example of the exercise status determination method in the exercise status determination apparatus according to the present embodiment. Here, descriptions of steps in FIG. 10 and FIG. 11 equivalent to those of the exercise status determination method depicted in FIG. 8 and FIG. 9 are simplified by reference to FIG. 8 and FIG. 9 as appropriate.

In a configuration where the extraction conditions depicted in FIG. 7 have been set, the processing for extracting sensor data and the like is broadly classified into four groups. In the following, the exercise status determination method including sensor data extracting processing is described for each group.

### First Example: In Cases where Extraction Conditions (1) to (4) are Specified

In (a first example of) the exercise status determination method according to the present embodiment, mainly, a presetting procedure, a sensing and data collection procedure, a sensor data extraction and transfer procedure, a data analyzing and processing procedure, and a data viewing and utilization procedure are sequentially performed, as depicted in the flowcharts of FIG. 8 and FIG. 9.

First, in the presetting procedure, as depicted in FIG. 8, the user US operates the input operation section 330 of the information communication terminal 300 to register each extraction condition item (refer to “condition item” depicted in FIG. 7) and details of each extraction condition (refer to “setting example” depicted in FIG. 7) which are applied when sensor data and the like are transferred to the network server 500 (Step S101). Here, when registering (1) distance or (2) time depicted in FIG. 7 as a condition for extracting sensor data and the like, the user US registers it by using a method of selecting a desired distance or a desired time interval (for example, every five minutes or every kilometer) from a setting screen displayed on the display section 340 or a method of directly inputting numerical values. When registering (3) pace change or (4) heart rate change as a condition for extracting sensor data and the like, the user US registers it by using a method of selecting a desired numerical value range (allowable range) or a desired change degree (amount of change within a predetermined time) of pace change or heart rate change from a setting screen displayed on the display section 340 or a method of directly inputting numerical values. When registering (5) altitude change or (6) temperature change as a condition for extracting sensor data and the like, the user US registers it by using a method of selecting a desired numerical value range (allowable range) or a desired change degree (amount of change within a predetermined time) of altitude change or temperature change from a setting screen displayed on the display section 340 or a method of directly inputting numerical values.

In the sensing and data collection procedure, as depicted in FIG. 8, the user US first operates the power supply switch of each of the wrist device 100 and the chest device 200 worn on the body to activate the wrist device 100 and the chest device 200 (Step S102). Next, by the user US operating the input interface section 130 of the wrist device 100 and the operation switch 230 of the chest device 200 simultaneously with or before or after the start of an exercise, the computation circuits 160 and 260 starts a sensing operation in the wrist device 100 and the chest device 200, respectively (Step S103). This sensing operation continues until the user US operates the input interface section 130 of the wrist device 100 and the operation switch 230 of the chest device 200 to end this operation simultaneously with or before or after the end of the exercise (Step S105). As a result, sensor data and the like indicating the movement status and the biological information of the user US during the exercise are collected (Step S104).

Specifically, in the wrist device 100 worn on the wrist of the user US, sensor data including acceleration data, angular velocity data, and geomagnetic data during the exercise such as running are detected by the sensor section 110, and GPS data including position data and movement speed data are detected by the GPS reception circuit 120, as depicted in FIG. 2A and FIG. 3. These detected data are each associated with time data and stored in the sensor data memory 171. Also, in the chest device 200 worn on the chest of the user US, sensor data including acceleration data, angular velocity data, and geomagnetic data during the running are detected by the sensor section 210, and heartbeat data is detected by the heartbeat detection circuit 220, as depicted in FIG. 2A and FIG. 4. These detected data are each associated with time data and stored in the sensor data memory 271. Furthermore, for example, in the wrist device 100, speed data (pace) is calculated by the computation circuit 160 based on the time data and the position data. Also, for example, in the chest device 200, a calorie consumption amount is calculated by the computation circuit 260 based on the time data, the heartbeat data, the weight and age of the user US, etc. These calculated data are each associated with time data and stored in the sensor data memories 171 and 271. Then, the sensor data, the GPS data, and the heartbeat data collected during the exercise, or the various information (the speed data, the calorie consumption amount, etc.) calculated based on the sensor data and the like are provided to the user US by, for example, being displayed on the display section 141 of the wrist device 100 in real time. Note that sensor data and heartbeat data obtained by the chest device 200 are transmitted to the wrist device 100 continuously or at predetermined time intervals by, for example, a wireless communication method such as Bluetooth (registered trademark) via the communication function section 250, and displayed on the display section 141.

In the sensor data extraction and transfer procedure, as depicted in FIG. 8, the wrist device 100 and the chest device 200 which have collected and stored the sensor data and the like are first connected to the information communication terminal 300 by a wireless communication method such as Bluetooth (registered trademark), or a wired communication method via a communication cable (Step S106). Next, the computation circuit 360 causes various extraction conditions registered in the presetting procedure to be displayed on the display section 340 of the information communication terminal 300. Then, the user US operates the input operation section 330 while viewing the display in order to select a desired condition item and its details from among the extraction conditions of (1) distance, (2) time, (3) pace change, and (4) heart rate change and determine various conditions for sensor data extraction processing (Step S107).

Next, a sensor data extraction request signal including the extraction condition specified by the user US (hereinafter simply referred to as a “request signal”) is transmitted from the information communication terminal 300 to the wrist device 100 and the chest device 200. Then, when the request signal is received in the wrist device 100 and the chest device 200, the computation circuits 160 and 260 each performs processing for extracting sensor data and the like matching the extraction condition from among the entire sensor data and the like stored in the sensor data memories 171 and 271, respectively (Step S108).

Specifically, in a case where (1) distance or (2) time depicted in FIG. 7 has been specified as the condition for extracting sensor data and the like, sensor data, GPS data, heartbeat data, speed data, a calorie consumption amount, and the like stored in the sensor data memories 171 and 271 at, for example, every kilometer or every five minutes during the exercise in association with time data are extracted. In a case where (3) pace change or (4) heart rate change has been specified as the extraction condition, sensor data, heartbeat data, a calorie consumption amount, and the like stored in association with time data corresponding to timing at which the pace or the heart rate has exceeded the numerical value range set in advance are extracted. Note that the sensor data and the like to be extracted herein may be single numerical value data corresponding to the timing (extraction point) matching the extraction condition, or may be numerical value data for a predetermined time period before and after the timing matching the extraction condition (for example, before the extraction point, after the extraction point, or for ten seconds around the extraction point).

Next, the extracted sensor data and the like (extracted data) are transmitted by the communication function sections 150 and 250 of the wrist device 100 and the chest device 200, respectively, to the information communication terminal 300, temporarily stored in the sensor data memory 371, and then transferred by the communication function section 350 of the information communication terminal 300 via the network 400 to the network server 500 (Step S109).

Note that the above-described sensor data extraction processing may be performed only on data having a relatively large data amount among sensor data detected by the sensor sections 110 and 210 of the wrist device 100 and the chest device 200, GPS data detected by the GPS reception circuit 120, heartbeat data detected by the heartbeat detection circuit 220, and speed data, a calorie consumption amount, and the like calculated by the computation circuits 160 and 260. For example, there is a case in which, in the above-described sensor data and the like, the data amount of the sensor data detected by the triaxial acceleration sensors 111 and 211, the triaxial angular velocity sensors 112 and 212, and the triaxial geomagnetic sensors 113 and 213 of the sensor sections 110 and 210 is several hundred to several thousand times larger compared with the data amount of the GPS data, the heartbeat data, and the like. Therefore, a configuration may be adopted in which, in a case like this, the above-described sensor data extraction processing based on an extraction condition is performed only on the sensor data detected by the sensor sections 110 and 210, and the processing result is transferred to the information communication terminal 300. In this configuration, the sensor data extraction processing is not performed on other data having a relatively small data amount, and the detected data (so-called raw data) is transferred as it is to the information communication terminal 300.

In the data analyzing and processing procedure, as depicted in FIG. 9, the computation circuit 560 of the network server 500 causes the sensor data and the like (transferred data) transferred by the information communication terminal 300 via the network 400 to be stored in the transfer data memory of the memory section 570. Next, the computation circuit 560 performs predetermined analysis and conversion processing based on the transferred data stored in the memory section 570 to generate analysis data and specific information regarding the exercise status of the user US. Specifically, in the analysis and conversion processing, the computation circuit 560 analyzes a movement route during the exercise of the user US and changes in the user's exercise form, heart rate, calorie consumption amount at the time of the extraction of the sensor data and the like in this movement route, in association with each other based on, for example, the GPS data include in the transferred data (Step S110). Here, regarding the exercise form of the user US, the computation circuit 560 judges the exercise status such as the pitch, the stride, the swing of the arms, the tilt of the body, the grounding time, the rhythm, the rotation of the hip, the ground reaction force, the spring model, and the swing of the legs based on the acceleration data, the angular velocity data, and the geomagnetic data included in the transferred data, and thereby generates form data (Step S111). This form data may be data processed as animation data with a skeleton model where the temporal change of the exercise form has been reflected. Also, the analysis data generated by the analysis processing may be data obtained by processing where changes of numerical values with respect to distance and time are made into a graph based on time data and distance data associated with the time data. These analysis data and specific information (the form data and the like) regarding the exercise status of the user US generated based on the analysis data are associated with map data indicating the movement route during the exercise and stored in a predetermined storage area of the database 600 (Step S112).

In the data viewing and utilization procedure, as depicted in FIG. 9, when the user US operates the information communication terminal 300 or the user terminal 700 to access the network server 500 via the network 400 or when the data analysis and conversion processing ends, the computation circuit 560 in the network server 500 reads out the analysis data and the specific information stored in the database 600, and processes the read analysis data and specific information into web display data having a predetermined display format. Next, the communication function section 550 transmits the processing results to the information communication terminal 300 and the user terminal 700 via the network 400. Then, the information communication terminal 300 and the user terminal 700 displays the web display data including the analysis data and the like transmitted via the network 400 on the display section 340 by using a web browser (Step S113). As a result, the user US can view the movement route, the analysis data, and the specific information displayed on the display section 340 of the information communication terminal 300 and a display section of the user terminal 700 singly or in a display format where these data have been coordinated with each other, and thereby can analyze his or her own exercise form and the like and reflect the analysis results in the improvement of an exercise method thereafter. (Step S114).

### Second Example: In a Case where Extraction Condition (5) or (6) is Specified

In a second example of the exercise status determination method according to the present embodiment, a series of processing depicted in FIG. 10 is performed in place of the sensor data extraction and transfer procedure (Steps S106 to S109) depicted in the flowcharts of FIG. 8 and FIG. 9. Specifically, extraction conditions for sensor data extraction processing are registered by the presetting procedure depicted in FIG. 8 (Step S101), and then sensor data and the like during the exercise of the user US are collected by the sensing and data collection procedure (Steps S102 to S105).

Next, in the sensor data extraction and transfer procedure, the wrist device 100 and the chest device 200 are connected to the information communication terminal 300 by a predetermined communication method, as depicted in FIG. 10 (Step S206). Then, the user US operates the information communication terminal 300 to select a desired condition item and details from among the extraction conditions of (5) altitude change and (6) temperature change and determine various conditions for sensor data extraction processing (Step S207).

Next, when a request signal including the extraction condition specified by the user US is transmitted from the information communication terminal 300 to the wrist device 100 and the chest device 200, the computation circuit 160 reads out position data included in the entire GPS data stored in the sensor data memory 171, and transmits the read position data and time data associated with the position data to the information communication terminal 300 (Step S208-1).

Next, the computation circuit 360 of the information communication terminal 300 causes the position data and the time data transmitted from the wrist device 100 to be stored in the sensor data memory 371, and causes the communication function section 350 to connect to the network 400 so as to obtain the altitude information of a position defined by each position data (latitude and longitude data) and the temperature information of this position at the time defined by the associated time data from a site or a dedicated server that provides environment information such as geographic information and meteorological information (Step S208-2). The obtained altitude information and temperature information are associated with the position data and the time data and stored in the sensor data memory 371.

Next, in the information communication terminal 300, the computation circuit 360 extracts altitude information or temperature information matching the selected and specified extraction condition from among the entire altitude information or temperature information stored in the sensor data memory 371, and reads out position data and time data associated with the altitude information or the temperature information.

Specifically, in a case where (5) altitude change or (6) temperature change depicted in FIG. 7 has been specified as a condition for extracting sensor data and the like, altitude information or temperature information acquired at, for example, timing (extraction point) at which the altitude change or the temperature change has exceeded a numerical value range set in advance is extracted, and time data associated with the altitude information or the temperature information is read out.

Next, a request signal including the time data read out corresponding to the altitude change or the temperature change is transmitted from the information communication terminal 300 to the wrist device 100 and the chest device 200. Then, when the request signal is received by the wrist device 100 and the chest device 200, the computation circuits 160 and 260 perform processing for extracting sensor data and the like associated with the time data corresponding to the extraction point from among the entire sensor data and the like stored in the sensor data memories 171 and 271 (Step S208-3). Note that the sensor data and the like to be extracted herein may be single numerical data associated with the time data matching the extraction condition, or may be numerical value data for a predetermined time period before and after the time (for example, before the extraction point, after the extraction point, or for ten seconds around the extraction point). As a result, sensor data, heartbeat data, a calorie consumption amount, and the like acquired at timing at which the altitude change or the temperature change has exceeded from a numerical value range set in advance are extracted.

Next, the extracted sensor data and the like (extracted data) are transmitted from the wrist device 100 and the chest device 200 to the information communication terminal 300, stored in the sensor data memory 371, and then transferred from the information communication terminal 300 via the network 400 to the network server 500 (Step S209).

Thereafter, as with the first example of the exercise status determination method, the data analyzing and processing procedure (Steps S110 to S112) and the data viewing and utilization procedure (Steps S113 to S114) depicted in FIG. 9 are performed.

### Third Example: In a Case where Extraction Method (7) is Applied

In a third example of the exercise status determination method according to the present embodiment, a series of processing depicted in FIG. 11 is performed in place of the sensing and data collection procedure (Steps S102 to S105) and the sensor data extraction and transfer procedure (Steps S106 to S109) depicted in the flowcharts of FIG. 8 and FIG. 9. Specifically, first, extraction conditions for sensor data extraction processing is registered by the pre-setting procedure depicted in FIG. 8 (Step S101).

Next, in the sensing and data collection procedure, the wrist device 100 and the chest device 200 are activated, as depicted in FIG. 11 (Step S302). Then, simultaneously with the start of an exercise of the user US or immediately before or after the start of the exercise, the computation circuits 160 and 260 starts a sensing operation in the wrist device 100 and the chest device 200 (Step S303), and thereby collects sensor data and the like indicating the movement status and the biological information of the user US during the exercise (Step S304). This sensing operation is continued until an end operation is performed simultaneously with the end of the exercise of the user US or immediately before or after the end of the exercise (Step S306). Here, by the user US operating the input interface section 130 of the wrist device 100 and the operation switch 230 of the chest device 200 at an arbitrary position or moment during the exercise so as to instructs to perform processing for extracting sensor data and the like, the timing of the extracting operation (extraction point) is stored in the sensor data memory 171 in association with time data (Step S305).

Next, in the sensor data extraction and transfer procedure, first, the wrist device 100 and the chest device 200 are connected to the information communication terminal 300 by a predetermined communication method, as depicted in FIG. 11 (Step S307). Next, in the wrist device 100, the computation circuit 160 reads out the time data stored in the sensor data memory 171 and associated with the extraction operation timing (extraction point) during the exercise, and transmits a request signal including the time data to the chest device 200 via the information communication terminal 300 or transmits it directly to the chest device 200. Accordingly, in the wrist device 100 and the chest device 200, the computation circuits 160 and 260 perform processing for extracting sensor data and the like associated with the time data corresponding to the extraction point from among the entire sensor data and the like stored in the sensor data memories 171 and 271 (Step S308). Note that the sensor data and the like to be extracted herein may be single numerical value data associated with the time data matching the extraction condition or numerical value data for a predetermined time period before and after the timing. As a result, sensor data, GPS data, heartbeat data, calorie consumption amount, and the like acquired at the timing desired by the user US are extracted.

Next, the extracted sensor data and the like (extracted data) are transmitted from the wrist device 100 and the chest device 200 to the information communication terminal 300, stored in the sensor data memory 371, and then transferred from the information communication terminal 300 to the network server 500 via the network 400 (Step S309).

Thereafter, as with the first example of the exercise status determination method, the data analyzing and processing procedure (Steps S110 to S112) and the data viewing and utilization procedure (Steps S113 to S114) depicted in FIG. 9 are performed.

In the above-described exercise status determination method, the processing procedure and the processing details according to each extraction condition for sensor data extraction processing have been described individually. However, sensor data extraction processing with a different extraction condition may be performed by combining the AND logic and the OR logic as appropriate.

(Specific Example of Sensor Data Extraction Processing)

Next, a specific example of sensor data extraction processing applied in the exercise status determination method according to the present embodiment is described with reference to the drawings.

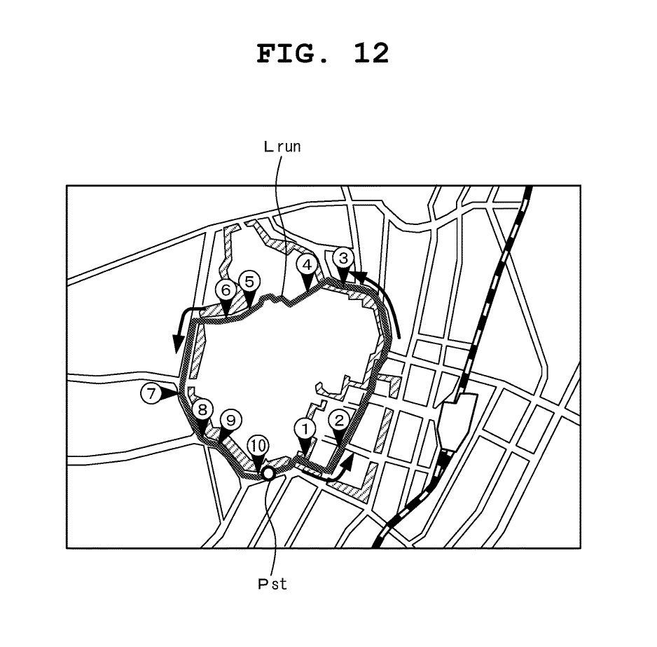

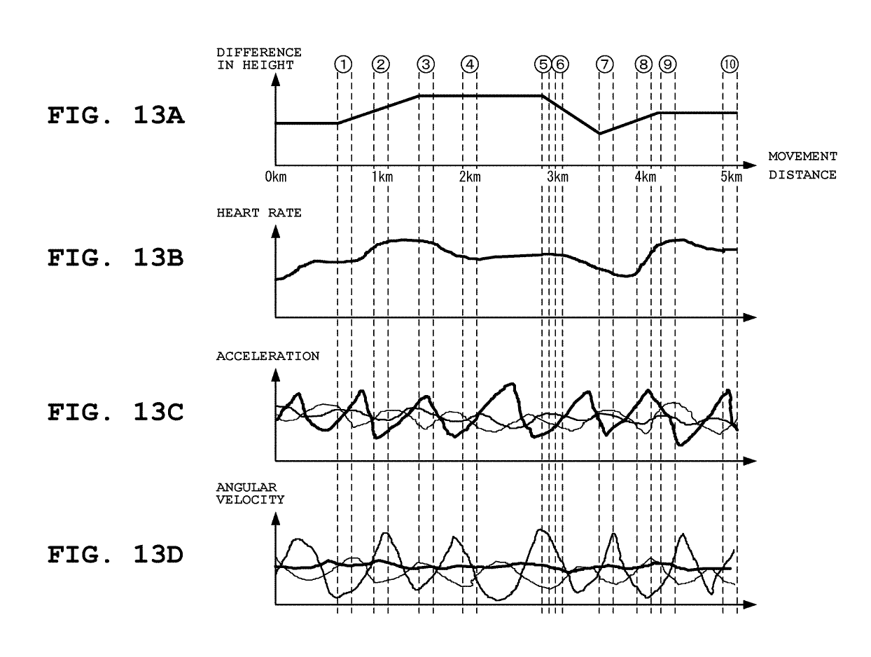

FIG. 12 is a schematic view depicting an example of a movement route of a user who is a target of sensor data extraction processing applied in the exercise status determination method according to the present embodiment. FIG. 13A to FIG. 13D are schematic views each depicting sensor data and the like obtained in the movement route depicted in FIG. 12 and the extraction points of the data. Here, numerals each surrounded by a circle and indicating an extraction point in the drawing are represented as “1” to “10”.

In this example, the user US moves by running or the like on a movement route (course) Lrun depicted in a map in FIG. 12 which has a difference in height as depicted in FIG. 13A. Pst in FIG. 12 represents a starting point of the movement route Lrun, that is, a start point of running.

As described in the above exercise status determination method, the user US runs on the movement route Lrun in FIG. 12 with the wrist device 100 and the chest device 200 being worn on his or her body, during which the sensor sections 110 and 210, the GPS reception circuit 120, the heartbeat detection circuit 220, and the like perform a sensing operation. As a result, sensor data (acceleration data, angular velocity data, and geomagnetic data), GPS data (position data and movement speed data), and heartbeat data are detected for each movement distance and elapsed time, and stored in the sensor data memories 171 and 271. These collected heartbeat data (heart rate), acceleration data, and angular velocity data for each movement distance are represented in the form of a graph as depicted in FIG. 13B, FIG. 13C and FIG. 13D, respectively.

Next, sensor data extraction processing is performed on the sensor data and the like stored in the sensor data memories 171 and 271, with an extraction condition specified by the user US or at timing specified by the user US during the running. For example, in a case where the user US desires to perform self analysis of his or her running form (exercise form), points where a difference in the height (for example, gradient) of the movement route Lrun is changed are specified as extraction points, which are “1”, “3”, “5”, and “7” in FIG. 13A and serve as extraction conditions thought to influence a change of the running form. By these extraction conditions being specified, sensor data and the like associated with the extraction points “1”, “3”, “5”, and “7” (specifically, time data associated with movement distances at the extraction points) are extracted in the sensor data extraction processing from among the sensor data and the like stored in the sensor data memories 171 and 271. Here, as depicted in FIG. 13A to FIG. 13D, sensor data and the like such as heartbeat data, acceleration data, and angular velocity data for the range of a predetermined distance (or a predetermined time) from the points at which a difference in the height is changed and which serve as the extraction points are extracted. These extracted sensor data and the like are transferred to the network server 500 via the information communication terminal 300.

Also, in a case where the user US desires to perform self analysis of his or her running form for each predetermined movement distance, points at every predetermined distance (for example, one kilometer) on the movement route Lrun are specified as extraction points, which are “2”, “4”, “6”, “8”, and “10” in FIG. 13A and serve as extraction conditions. By specifying these extraction conditions, sensor data and the like associated with the extraction points “2”, “4”, “6”, “8” and “10” are extracted from among the sensor data and the like stored in the sensor data memories 171 and 271. Here, as depicted in FIG. 13A to FIG. 13D, sensor data and the like such as heartbeat data, acceleration data, and angular velocity data for the range of a predetermined distance (or a predetermined time) from the predetermined movement distance points that serve as the extraction points are extracted. These extracted sensor data and the like are transferred to the network server 500 via the information communication terminal 300.

(Display Example of Exercise Information)

Next, with reference to the drawings, description is made to a display example of analysis data and the like that are generated by the network server 500 and displayed on the user terminal 700 or the information communication terminal 300 in the exercise status determination method according to the present embodiment.

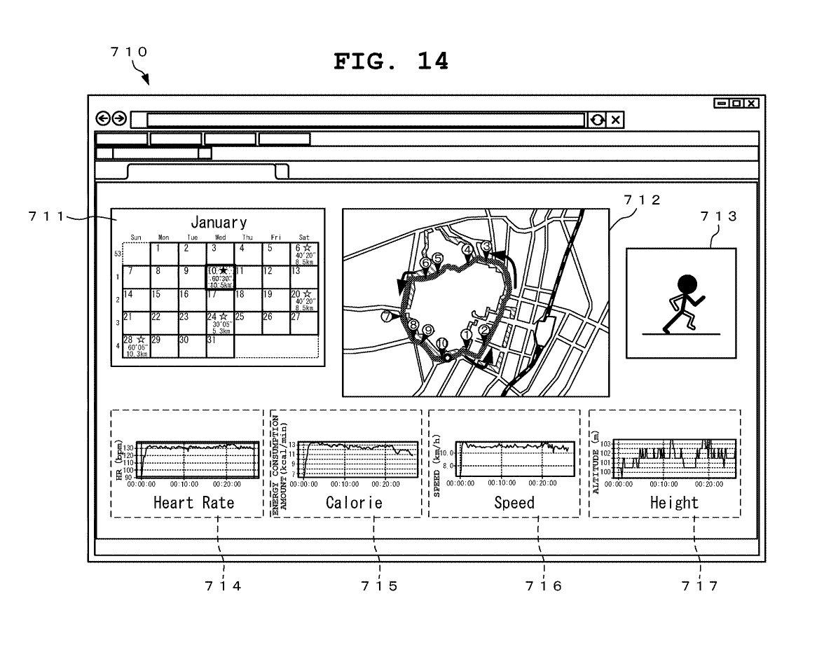

FIG. 14 is a schematic view depicting a display example of analysis data and the like that are displayed on a user terminal or the like applied in the exercise status determination apparatus according to the present embodiment.

As described above, the user terminal 700 and the information communication terminal 300 each include a function for connecting to the network 400 such as the Internet, and each have incorporated therein a web browser as viewing software. Therefore, by accessing the network server 500 via the network 400, the user terminal 700 and the like can receive web display data including analysis data and the like generated by analyzing sensor data and the like in the network server 500, and display the web display data on, for example, a web screen 710 having a predetermined display format on the display section, as depicted in FIG. 14. Here, a display example when a personal computer is adopted as the user terminal 700 and the like is depicted.

On the web screen 710 displayed on the display section of the user terminal 700 and the like, for example, a calendar 711 indicating the date and time of running and their details, a map 712 indicating a running route (movement route), and a skeleton animation 713 indicating a running form are placed in the middle, and a heartbeat data graph 714, a calorie consumption amount graph 715, a running speed graph 716, and an altitude graph 717 indicating altitudes at running points are placed on the bottom, as depicted in FIG. 14. This display is achieved by performing predetermined processing on analysis data generated by the network server 500 and specific information regarding the exercise status of the user US generated based on the analysis data, such as by making the data into graphs, capturing the data into map information, and making the data into animation. Also, the analysis data and the specific information for use in this display have been associated with each other. For example, by specifying an arbitrary point on the running route in the map with a mouse pointer, a touch panel, or the like, positions in the graphs 714 to 717 corresponding to that point are displayed, and the movement of the skeleton animation 713 is displayed in conjunction with this specification. As a result, the user US can view the map 712, the skeleton animation 713, the graphs 714 to 717, and the like displayed on the web screen 710 in cooperation with each other as appropriate, and can perform self analysis of his or her exercise status, running form, and the like to reflect the analysis results in the improvement of an exercise method thereafter.

As described above, in the present embodiment, by a desired extraction condition being specified to perform extraction processing for sensor data and the like obtained by the wrist device 100, the chest device 200, and the like, sensor data matching the extraction condition and associated with each other is extracted and transferred to the network server 500 via the information communication terminal 300. As a result of this configuration, from among entire sensor data and the like obtained by the wrist device 100 and the chest device 200, for example, only sensor data and the like matching an extraction condition through to influence a change of the exercise form of the user US or sensor data and the like acquired at arbitrary timing desired by the user US can be selectively extracted and used for analysis processing in the network server 500.

Therefore, according to the present embodiment, the amount of data that is transferred from the sensor devices of the wrist device 100, the chest device 200, and the like to the network server 500 via the information communication terminal 300 can be significantly reduced compared with an entire data amount stored in the sensor data memories 171 and 271. Accordingly, the data transfer time can be reduced, and power consumption required at the time of the data transfer can also be reduced. Also, in the present embodiment, since the amount of data that is transferred from the sensor devices can be reduced, the storage capacity of memories included in the information communication terminal 300 and the network server 500 can be reduced, and the product cost can be reduced. Moreover, by the configuration where transferred sensor data and the like is analyzed and processed by the network server 500 connected to the network 400, processing load on the sensor devices and the information communication terminal 300 can be reduced, and the analysis and processing of sensor data and the like having a large data amount can be quickly performed.

Next, modification examples of the above-described embodiment are described.

### First Modification Example

In the above-described embodiment, in the wrist device 100 and the chest device 200, entire sensor data and the like (raw data) detected by the sensor sections 110 and 210, the GPS reception circuit 120, and the heart rate detection circuit 220 during an exercise is stored in the sensor data memories 171 and 271, and only sensor data and the like matching an extraction condition specified by the information communication terminal 300 is extracted from among the entire sensor data and the like after the end of the exercise, and transferred to the network server 500 via the information communication terminal 300.

However, the present invention is not limited thereto, and a configuration may be adopted in which a desired extraction condition is specified in advance in the wrist device 100 and the chest device 200, sensor data and the like detected by the sensor sections 110 and 210 and the like during an exercise are stored in the sensor data memories 171 and 271 only when the extraction condition is satisfied, and only the extracted and stored sensor data and the like are transferred via the information communication terminal 300 to the network server 500 after the end of the exercise.

With this configuration, sensor data and the like detected by the sensor sections 110 and 210, the GPS reception circuit 120, and the heart rate detection circuit 220 are stored in the sensor data memories 171 and 271 after extraction processing. Therefore, the data amount of sensor data and the like can be significantly reduced, and the storage capacity of the sensor data memories 171 and 271 can be reduced.

### Second Modification Example

In the above-described embodiment, analysis processing is performed by using only sensor data and the like (transferred data) obtained in the wrist device 100 and the chest device 200 and transferred to the network server 500 via the information communication terminal 300, and the analysis data and the specific information are displayed on the display section of the user terminal 700 and the information communication terminal 300.

However, the present invention is not limited thereto, and a configuration may be adopted in which a request for verification of sensor data and the like transferred to the network server 500, their analysis data, and the specific information is made to an expert (for example, a coach or an instructor), and an advice from the expert and the like is displayed together with the analysis data and the specific information on the display section of the user terminal 700 and the information communication terminal 300.

With this configuration, the user's own exercise status, exercise form, and the like can be more accurately determined with reference to an advice from an expert and the like, which can be reflect in the improvement of an exercise method thereafter.

### Third Modification Example

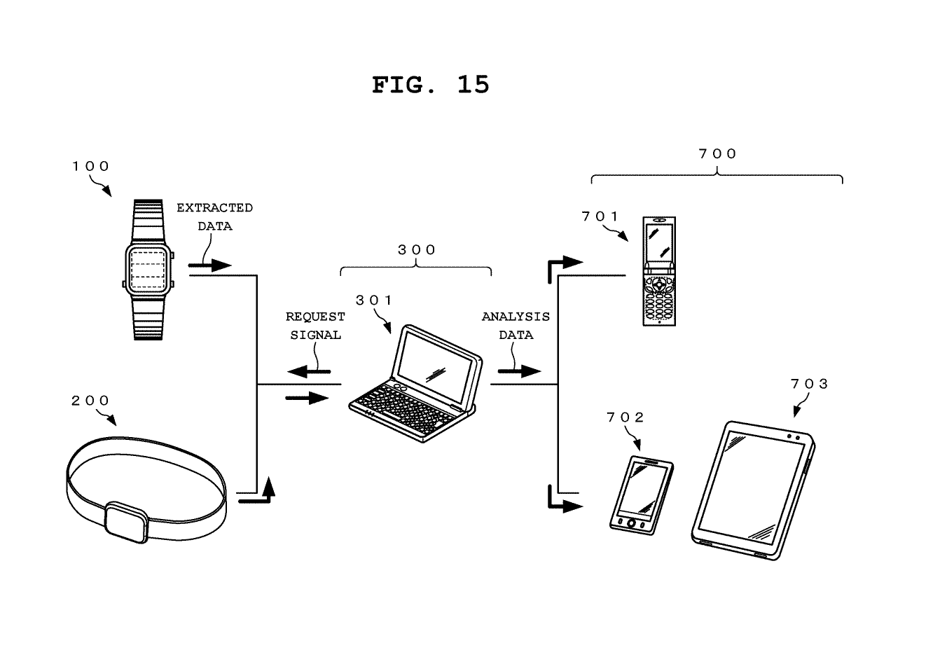

FIG. 15 is a schematic structural view depicting a modification example of the exercise status determination apparatus according to the above-described embodiment. Here, components similar to those of the above-described embodiment (refer to FIG. 1) are provided with the same reference numeral and their descriptions are simplified.

The above-described embodiment includes a so-called cloud-computing-type system where sensor data and the like obtained by the wrist device 100 and the chest device 200 are transferred via the information communication terminal 300 to the network server 500 connected to the network 400, analyzed and processed by the network server 500, and then provided to the user terminal 700.

However, the present invention is not limited thereto, and a configuration may be adopted in which sensor data and the like obtained by the wrist device 100 and the chest device 200 are analyzed and processed directly in the information communication terminal 300 and then provided to the user terminal 700.

Specifically, an exercise status determination apparatus according to the present modification example mainly has the wrist device 100 and the chest device 200, the information communication terminal 300, and the user terminal 700 as depicted in FIG. 15, and the information communication terminal 300 includes a processing function similar to the processing for analyzing and processing sensor data and the like which is performed in the network server 500 in the above-described embodiment.

In this exercise status determination apparatus, first, sensor data and the like are obtained by the wrist device 100 and the chest device 200, a request signal that specifies a desired extraction condition is transmitted from the information communication terminal 300 to the wrist device 100 and the chest device 200, and whereby only sensor data and the like matching the extraction condition is extracted and transferred to the information communication terminal 300. Subsequently, in the information communication terminal 300, the transferred sensor data and the like are analyzed and processed to generate analysis data and specific information based on the analysis data. Then, the analysis data and the specific information are displayed in a predetermined display format on the display section 340 of the information communication terminal 300. Also, the analysis data and the specific information may be transmitted to the user terminal 700 such as the portable telephone 701, the smartphone 702, or the tablet terminal 703 connected to the information communication terminal 300 by a predetermined communication method, and displayed in a predetermined display format on the display section of the user terminal 700. In this case, as a method for transmitting the analysis data and the like from the information communication terminal 300 to the user terminal 700, the information communication terminal 300 and the user terminal 700 may be directly connected to each other via wireless communication, infrared communication, a communication cable, or the like. Alternatively, the data transmission may be performed via a network such as a portable telephone network or the Internet, or via a memory card or the like.

In this configuration, sensor data and the like obtained in the wrist device 100 and the chest device 200 are transferred to the information communication terminal 300, and the analysis and processing thereof is performed in the information communication terminal 300. Therefore, the time required for transferring sensor data and the like can be reduced. Also, depending on the extraction condition specified by the information communication terminal 300 (in the case of an extraction condition other than altitude change extraction and temperature change extraction), a network connection environment is not required. Therefore, even in a situation where the information communication terminal 300 does not include a network connection function or connection cannot be made to a network, the analysis and processing of sensor data and the like and the generation of analysis data and the like can be performed, and appropriate information can be provided to the user.

In FIG. 15, as the information communication terminal 300 that analyzes and processes sensor data and the like transferred from the wrist device 100 and the chest device 200, the personal computer 301 with a relatively high computation capability has been adopted from among the information communication terminals 300 depicted in FIG. 1. However, another terminal such as a smartphone or a tablet terminal may be adopted, depending on the details of computation processing or when the terminal has a high computation capability.

### Fourth Modification Example

In the above-described embodiment, an extraction condition for extracting sensor data and the like obtained in the wrist device 100 and the chest device 200 is specified by the information communication terminal 300 and a request signal is transmitted to the wrist device 100 and the chest device 200 to perform sensor data extraction processing.

However, the present invention is not limited thereto, and a configuration may be adopted in which an extraction condition is specified by the wrist device 100 including a display section, extraction processing is performed on sensor data and the like obtained in the wrist device 100, and a request signal including the extraction condition is transmitted to the chest device 200 to perform extraction processing on sensor data and the like obtained in the chest device 200.

With this configuration, the operation for obtaining sensor data and the like and the processing for extracting sensor data and the like matching a desired extraction condition can be performed only by a sensor device worn on the body of the user US. Therefore, the procedure of specifying a condition for extracting sensor data and the like in the information communication terminal 300 can be omitted. Also, the operation of transferring sensor data and the like via the information communication terminal 300 can be quickly started after the end of the exercise. As a result, processing load on the information communication terminal 300 can be reduced, and the usability of the exercise status determination apparatus can be improved.

In the embodiment and the modification examples described above, the wrist device 100 that is worn on a wrist and the chest device 200 that is worn on a chest have been adopted as sensor devices in the present invention. However, the present invention is not limited thereto, and another sensor device may be adopted as long as it can obtain sensor data and the like indicating the motion status and the biological information of the human-body during an exercise. For example, a sensor device that is worn on an upper arm, an ankle, a hip, a shoelace, or the like may be adopted.

Also, in the embodiment described above, running is exemplarily described as an exercise to which the exercise determination device is applied. However, the present invention is not limited thereto and may be applied to various exercises, such as walking, cycling, trekking, and mountaineering.

While the present invention has been described with reference to the preferred embodiments, it is intended that the invention be not limited by any of the details of the description therein but includes all the embodiments which fall within the scope of the appended claims.

What is claimed is:

1. A sensor data extraction system comprising:
at least one computation circuit; and
at least one memory storing instructions that, when executed by the at least one computation circuit, cause the at least one computation circuit to perform operations including:
obtaining exercise data including position data of a human body during exercise;
specifying an extraction condition for acquiring exercise data for analysis from the obtained exercise data, the extraction condition relating to a meteorological change;
obtaining environmental information including meteorological information from environmental information stored in a storage, the environmental information being associated with time data;
extracting environmental information from the obtained environmental information, based on the specified extraction condition relating to the meteorological change; and
acquiring the exercise data for analysis based on the associated time data of the extracted environmental information.

2. The sensor data extraction system according to claim 1, wherein the at least one computation circuit obtains the environmental information from a server via a network, the server comprising the storage.

3. The sensor data extraction system according to claim 1, wherein the at least one computation circuit further specifies, as a further extraction condition, at least one of a movement distance of the human body, an elapsed time, a heart rate change, a movement speed change, and a geographical change.

4. The sensor data extraction system according to claim 1, further comprising an exercise sensor device which obtains the exercise data;
wherein the exercise sensor device obtains at least exercise data related to an exercise form of the human body.

5. The sensor data extraction system according to claim 4, wherein the exercise sensor device obtains acceleration data, angular velocity data, and geomagnetic data each having triaxial components as the exercise data related to the exercise form of the human body.

6. The sensor data extraction system according to claim 1, further comprising an exercise sensor device which obtains the exercise data;
wherein the exercise sensor device obtains exercise data including biological information of the human body.

7. The sensor data extraction system according to claim 1, further comprising an exercise sensor device which obtains the exercise data;
wherein the exercise sensor device obtains exercise data related to a position and a movement speed of the human body.

8. The sensor data extraction system according to claim 1, the operations performed by the at least one computation circuit further comprising transferring the acquired exercise data to outside of a device that acquired the exercise data.

9. The sensor data extraction system according to claim 1, further comprising at least one exercise sensor which obtains the exercise data, wherein the at least one computation circuit, the at least one memory, and the at least one exercise sensor are provided in a same sensor device.

10. The sensor data extraction system according to claim 8, further comprising a data analysis device;
wherein the transferring comprises transferring the acquired exercise data to the data analysis device via a network.

11. The sensor data extraction system according to claim 1, the operations performed by the at least one computation circuit further comprising analyzing the acquired exercise data.

12. The sensor data extraction system according to claim 10, the operations performed by the at least one computation circuit further comprising:
providing analysis data generated by the data analysis device to a user.

13. A sensor data extracting method comprising:
obtaining exercise data including position data of a human body during exercise;
specifying an extraction condition for acquiring exercise data for analysis from the obtained exercise data, the extraction condition relating to a meteorological change;
obtaining environmental information including meteorological information from environmental information stored in a storage, the environmental information being associated with time data;
extracting environmental information from the obtained environmental information, based on the specified extraction condition relating to the meteorological change; and
acquiring the exercise data for analysis based on the associated time data of the extracted environmental information.

14. A non-transitory computer-readable storage medium having stored thereon a sensor data extraction program that is executable by a computer, the program being executable by the computer to perform functions comprising;
obtaining exercise data including position data of a human body during exercise;
specifying an extraction condition for acquiring exercise data for analysis from the obtained exercise data, the extraction condition relating to a meteorological change;
obtaining environmental information including meteorological information from environmental information stored in a storage, the environmental information being associated with time data;
extracting environmental information from the obtained environmental information, based on the specified extraction condition relating to the meteorological change; and
acquiring the exercise data for analysis based on the associated time data of the extracted environmental information.
